(19) United States
(12) Patent Application Publication
(10) Pub. No.: US 2015/0224129 A1
(43) Pub. Date: Aug. 13, 2015

(54) METHODS AND PHARMACEUTICAL COMPOSITIONS FOR THE PROPHYLACTIC TREATMENT OF BACTERIAL SUPERINFECTIONS POST-INFLUENZA WITH INVARIANT NKT CELL AGONISTS

(71) Applicants: INSERM (Institut National de la Santé et de la Recherche Médicale, Paris (FR); INSTITUT PASTEUR DE LILLE, Lille (FR)

(72) Inventors: Francois Trottein, Lille (FR); Christelle Faveeuw, Lille (FR); Stoyan Ivanov, Lille (FR); Josette Fontaine, Lille (FR)

(73) Assignees: INSERM (Institut National de la Santé et de la Recherche Médicale), Paris (FR); INSTITUT PASTEUR DE LILLE, Lille (FR); UNIVERSITÉ DE LILLE 1 - SCIENCES ET TECHNOLOGIES, VILLENEUVE-D'ASCQ (FR); UNIVERSITÉ DU DROIT ET DE LA SANTÉ LILLE 2, LILLE (FR); CENTRE NATIONAL DE LA RECHERCHE SCIENTIFIQUE (CNRS), Paris (FR)

(21) Appl. No.: 14/430,375

(22) PCT Filed: Oct. 1, 2013
(86) PCT No.: PCT/EP2013/070441

§ 371 Date: Mar. 23, 2015

Related U.S. Application Data
(60) Provisional application No. 61/773,009, filed on Mar. 5, 2013.

(30) Foreign Application Priority Data
Oct. 3, 2012 (EP) 12306205.1

(51) Int. Cl. A61K 31/7028; A61K 45/06

(52) U.S. Cl. CPC A61K 31/7028; A61K 45/06

(57) ABSTRACT

The present invention relates to methods and pharmaceutical compositions for the prophylactic treatment of bacterial superinfections post-influenza with iNKT cell agonists.

### FIELD OF THE INVENTION

The present invention relates to methods and pharmaceutical compositions for the prophylactic treatment of bacterial superinfections post-influenza with iNKT cell agonists.

### BACKGROUND OF THE INVENTION

Influenza A virus (IAV) infection is one of the most important causes of respiratory tract diseases and is responsible for widespread morbidity and mortality (1). During the first several days after infection, the host develops a complex and effective innate immune response that allows to contain IAV replication pending the development of adaptive immune responses (2, 3). However, at later time points, increased susceptibility to bacterial superinfection can occur leading to mortality during IAV epidemics and pandemics (4-6). For instance bacterial pneumonias accounted for the majority of deaths (˜20-40 million deaths worldwide) in the 1918 pandemic (Spanish flu) (7). Among the predominant bacteria species causing bacterial superinfection post-IAV are Streptococcus pneumoniae (the pneumococcus), Haemophilus influenzae and Staphylococcus aureus (5). Although there are evidences of specific features of individual types of bacteria, the mechanisms leading to enhanced susceptibility to secondary bacterial infection seem to be broad-based and include alterations of mechanical and immunological defences (8-10). Indeed, alteration of the physical barriers to bacterial adhesion and invasion including alteration of the mucosa as well as the exposition of new attachment sites for the bacteria have been described. In parallel, impairment of the host innate (rather than adaptive) response is a cardinal feature of bacterial-associated pneumonia post-influenza challenge (11). The contribution of invariant Natural Killer T (iNKT) cells in bacterial superinfections post influenza has not yet been apprehended.

Invariant NKT cells represent a population of “innate-like” TCRαβ lymphocytes that conduct important immunostimulatory and regulatory functions, particularly during infection (for reviews, (12-14)). These cells express a conserved T cell receptor which recognizes self and exogenous (glyco)lipids presented by the CD1d molecule (for reviews, (15-19)). In response to TCR triggering, as well as to certain stress-induced cytokines, iNKT cells promptly produce a wide array of cytokines including IFN-γ, IL-4 and/or IL-17. This cytokine burst is crucial to trans-activate other cells of the innate and adaptive systems. Although not abundant in the lung tissue, iNKT cells appear to act as key players in mucosal immunity that takes place in this site. In particular, they can actively participate in host defence against respiratory viral and bacterial pathogens (13, 14). On the other hand, during infection (20) and in sterile (20-24) conditions, iNKT cells can also strongly participate in lung inflammation and decreased pulmonary functions.

### SUMMARY OF THE INVENTION

The present invention relates to an iNKT cell agonist for use in the prophylactic treatment of bacterial superinfections post-influenza in a subject in need thereof.

### DETAILED DESCRIPTION OF THE INVENTION

Influenza A virus (IAV) predisposes to virulent bacterial (i.e. Streptococcus pneumoniae) infections, which account for much of the mortality during IAV seasonal epidemics and pandemics. The innate immune response plays an important role in staving off S. pneumoniae infection but mechanisms of immunosupression in IAV-experienced hosts hinder productive anti-pneumococcal innate immunity. The inventors investigated the potential role of invariant Natural Killer T (iNKT) cells, a population of potent immunoregulatory cells, in experimental bacterial superinfection post-IAV challenge. They show that whilst iNKT cells control S. pneumoniae serotype 1 infection in single infected mice, they failed to do so in mice previously infected with IAV (H3N2). This later effect is associated with a lack of iNKT cell activation (IFN-γ production) at the time of S. pneumoniae challenge. Neutralizing of IL-10, the production of which peaking at day 7 post-IAV challenge, restored the activation of iNKT cells during S. pneumoniae challenge as well as host defences against the bacteria. Next, the inventors examined whether direct exogenous stimulation of iNKT cells with the superagonist α-galactosylceramide can reverse the influenza induced immunosuppression to control S. pneumoniae. They show that administration of an iNKT cell agonist (i.e. α-galactosylceramide) protects against bacterial superinfection post-influenza.

Accordingly, the present invention relates to an iNKT cell agonist for use in the prophylactic treatment of bacterial superinfections post-influenza in a subject in need thereof.

The subject can be human or any other animal (e.g., birds and mammals) susceptible to influenza infection (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal or pet. In another embodiment, a subject is a human.

According to the invention the subject has an influenza infection. As used herein, the term “influenza infection” has its general meaning in the art and refers to the disease caused by an infection with an influenza virus. In some embodiments of the invention, influenza infection is associated with Influenza virus A or B. In some embodiments of the invention, influenza infection is associated with Influenza virus A. In some specific embodiments of the invention, influenza infection is cause by influenza virus A that is H1N1, H2N2, H3N2 or H5N1.

The terms “prophylaxis” or “prophylactic use” and “prophylactic treatment” as used herein, refer to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease.

The prophylactic methods of the invention are particularly suitable for the prevention of bacterial superinfections post-influenza such as, but not limited to infections of the lower respiratory tract (e.g., pneumonia), middle ear infections (e.g., otitis media) and bacterial sinusitis. The bacterial superinfection may be caused by numerous bacterial pathogens. For example, they may be mediated by at least one organism selected from the group consisting of Streptococcus pneumoniae, Staphylococcus aureus, Haemophilus influenza, Myoplasma species and Moraxella catarrhalis.

The prophylactic methods of the invention are particularly suitable for subjects who are identified as at high risk for developing a bacterial superinfection post-influenza, including subjects who are at least 50 years old, subjects who reside in chronic care facilities, subjects who have chronic disorders of the pulmonary or cardiovascular system, subjects who required regular medical follow-up or hospitalization during the preceding year because of chronic metabolic diseases (including diabetes mellitus), renal dysfunction, hemoglobinopathies, or immunosuppression (including immunosuppression caused by medications or by human immunodeficiency (HIV) virus); children less than 14 years of age, patients between 6 months and 18 years of age who are receiving long-term aspirin therapy, and women who will be in the second or third trimester of pregnancy during the influenza season. More specifically, it is contemplated that the prophylactic method of the invention are suitable for the prevention of bacterial superinfections post-influenza in subjects older than 1 year old and less than 14 years old (i.e., children); subjects between the ages of 50 and 65, and adults who are older than 65 years of age.

As used herein, the term “iNKT cell agonist” has its general meaning in the art and refers to any derivative or analogue derived from a lipid, that is typically presented in a CD1d context by antigen presentating cells (APCs) and that can promote, in a specific manner, cytokine production by iNKT cells. Typically the iNKT cell agonist is a α-galactosylceramide compound.

As used herein, the term “α-galactosylceramide compound” or “α-GalCer compound” has its general meaning in the art and refers to any derivative or analogue derived from a glycosphingolipid that contains a galactose carbohydrate attached by an α-linkage to a ceramide lipid that has an acyl and sphingosine chains of variable lengths (Van Kaer L. α-Galactosylceramide therapy for autoimmune diseases: Prospects and obstacles. Nat. Rev. Immunol. 2005; 5: 31-42).

Various publications have described α-galactosylceramide compounds and their synthesis. An exemplary, but by no means exhaustive, list of such references includes Morita, et al., J. Med. Chern., 25 38:2176 (1995); Sakai, at al., J. Med. Chern., 38:1836 (1995); Morita, et al., Bioorg. Med. Chem. Lett., 5:699 (1995); Takakawa, et al., Tetrahedron, 54:3150 (1998); Sakai, at al., Org. Lett., 1:359 (1998); Figueroa-Perez, et al., Carbohydr. Res., 328:95 (2000); Plettenburg, at al., J. Org. Chern., 67:4559 (2002); Yang, at al., Angew. Chem., 116:3906 (2004); Yang, at al., Angew. Chern. Int. Ed., 43:3818 (2004); and, Yu, et al., Proc. Natl. Acad. Sci. USA, 102(9):3383-3388 (2005).

Examples of patents and patent applications describing instances of α-galactosylceramide compounds include U.S. Pat. No. 5,936,076; U.S. Pat. No. 6,531,453 U.S. Pat. No. 5,S53,737, U.S. Pat. No. 8,022,043, US Patent Application 2003030611, US Patent Application 20030157135, US Patent Application 20040242499, US Patent Application 20040127429, US Patent Application 20100104590, European Patent EP0609437 and International patent application WO2006026389.

A typical α-galactosylceramide compound is KRN7000 ((2S 3S, 4R)-1-0-(alfaD-galactopyranosyl)-N-hexacosanoyl-2-amino-1,3,4-octadecanetriol)) (KRN7000, a novel immunomodulator, and its antitumor activities. Kobayashi E, Motoki K, Uchida T, Fukushima H, Koezuka Y. Oncol Res. 1995;7(10-11):529-34.).

Other examples includes:

(2S,3R)-1-(a-D-galactopyranosyloxy)-2-tetracosanoylamino-3-octadecanol,

(2S,3R)-2-docosanoylamina-1-(a-Dgalactopyranosyloxy)-3-octadecanol,

(2S,3R)-1-(a-D-galactopyranosyloxy)-2-icosanoylamino-3-octadecanol,

(2S,3R)-1-(a-D-galactopyranosyloxy)-2-octadecanoylamino-3-octadecanol,

(2S,3R)-1-(a-D-galactopyranosyloxy)-2-tetradecanoylamino-3-octadecanol,

(2S,3R)-2-decanoylamino-1-(a-D-40galactopyranosyloxy)-3-octadecanol,

(2S,3R)-1-(a-D-galactopyranosyloxy)-2-tetracosanoylamino-3-tetradecanol,

(2S,3R)-1-(a-D-galactopyranosyloxy)-2-tetradecanoylamino-3-hexadecanol,

(2R,3S) -1-(a-D-galactopyranosyloxy)-2-tetradecanoylamino-3-hexadecanol,

(2S,3S) -1-(a-D-galactopyranosyloxy)-2-tetradecanoylamino-3-hexadecanol,

(2S,3R)-1-(a-D-galactopyranosyloxy)-2[(R)-2-hydroxytetracosanoylamino]-3-octadecanol,

(2S,3R,4E)-1-(a-D-galactopyranosyloxy)-2-octadecanoylamino-4-octadecen-3-ol,

(2S,3R,4E)-1-(a-D-galactopyranosyloxy)-2-tetradecanoylamino-4-octadecen-3-ol,

(2S,3S,4R)-1-(a-D-galactopyranosyloxy)-2-tetracosanoylamino-3,4-octadecanediol,

(2S,3S,4R)-1-(a-D-galactopyranosyloxy)-2-tetracosanoylamino-3,4-heptadecanediol,

(2S,3S,4R)-1-(a-D-galactopyranosyloxy)-2-tetracosanoylamino-3,4-pentadecanediol,

(2S,3S,4R)-1-(a-D-galactopyranosyloxy)-2-tetracosanoylamino-3,4-undecanediol,

(2S,3S,4R)-1-(a-D-galactopyranosyloxy)-2-hexacosanoylamino-3,4-heptadecanediol,

(2S,3S,4R)-1-(a-D-galactopyranosyloxy)-2-[(R)-2-hydroxytetracosanoylamino]-3,4-octadecanediol,

(2S,3S,4R)-1-(a-D-galactopyranosyloxy)-2-[(R)-2-hydroxytetracosanoylamino]-3,4-heptadecanediol,

(2S,3S,4R)-1-(a-D-galactopyranosyloxy)-2-[(R)-2-hydroxytetracosanoylamino]-3,4-pentadecanediol,

(2S,3S,4R)-1-(a-D-galactopyranosyloxy)-2-[(R)-2-hydroxytetracosanoylamino]-3,4-undecanediol,

(2S,3S,4R)-1-(a-D-galactopyranosyloxy)-2-[(R)-2-hydroxyhexacosanoylamino]-3,4-octadecanediol,

(2S,3S,4R)-1-(a-D-galactopyranosyloxy)-2-[(R)-2-hydroxyhexacosanoylamino]-3,4-nonadecanediol,

(2S,3S,4R)-1-(a-D-galactopyranosyloxy)-2-[(R)-2-hydroxyhexacosanoylamina]-3,4-icosanediol,

(2S,3S,4R)-1-(a-D-galactopyranosyloxy)-2-[(S)-2-hydroxytetracosanoylamino]-3,4-heptadecanediol,

(2S,3S,4R)-1-(a-D-galactopyranosyloxy)-2-[(R)-2-hydroxytetracosanoylamino]-3,4-hexadecanediol,

(2S,3S,4R)-1-(a-D-galactopyranosyloxy)-2-[(S)-2-hydroxytetracosanoylamino]-16-methyl-3,4-heptadecanediol,

(2S,3S,4R)-1-(a-D-galactopyranosyloxy)-16-methyl-2-tetracosanoylamino-3,4-heptadecanediol,

(2S,3S,4R)-1-(a-D-galactopyranosyloxy)-2-[(R)-2-hydroxytricosanoylamino]-16-methyl-3,4-heptadecanediol,

(2S,3S,4R)-1-(a-D-galactopyranosyloxy)-2-[(R)-2-hydroxypentacosanoylamino]-16-methyl-3,4-octadecanediol,

(2S,3R)-1-(a-D-galactopyranosyloxy)-2-oleoylamino-3-octadecanol,

(2S,3S,4R)-1-(a-D-galactopyranosyloxy)-2-hexacosanoylamino-3,4-octadecanediol;

(2S,3S,4R)-1-(a-D-galactopyranosyloxy)-2-octacosanoylamino-3,4-heptadecanediol

(2R,3R)-1-(a-D-galactopyranosyloxy)-2-tetradecanoylamino-3-hexadecanol

(2S,3R,4S,5R)-2-((2S,3S,4R)-2-(4-hexyl-1H-1,2,3-triazol-1-yl)-3,4-dihydroxyoctadecyloxy)-6-(hydroxymethyl)-tetrahydro-28- pyrane-3,4,5-triol;

(2S,3R,4S,5R)-2-((2S,3S,4R)-2-(4-heptyl-1H-1,2,3-triazol-1-yl)-3,4-dihydroxyoctadecyloxy)-6-(hydroxymethyl)-tetrahydro-28- pyrane-3,4,5-triol;

(2S,3R,4S,5R)-2-((2S,3S,4R)-2-(4-hexadecyl-1H-1,2,3-triazol-1-yl)-3,4-dihydroxyoctadecyloxy)-6-(hydroxymethyl)-tetrahydro-28-pyrane-3,4,5-triol;

(2S,3R,4S,5R)-2-((2S,3S,4R)-3,4-dihydroxy-2-(4-tricosyl-1H-1,2,3-triazol-1-yl)octadecyloxy)-6-(hydroxymethyl)-tetrahydro-28-pyrane-3,4,5-triol;

(2S,3R,4S,5R)-2-((2S,3S,4R)-3,4-dihydroxy-2-(4-tetracosyl-1H-1,2,3-triazol-1-yl)octadecyloxy)-6-(hydroxymethyl)-tetrahydro-2H-pyrane-3,4,5-triol;

(2S,3R,4S,5R)-2-((2S,3S,4R)-3,4-dihydroxy-2-(4-pentacosyl-1H-1,2,3-triazol-1-yl)octadecyloxy)-6-(hydroxymethyl)-tetrahydro-28-pyrane-3,4,5-triol;

(2S,3R,4S,5R)-2-((2S,3S,4R)-3,4-dihydroxy-2-(4-(6-phenylhexyl)-1H-1,2,3-triazol-1-yl)octadecyloxy)-6-(hydroxymethyl)- tetrahydro-28-pyrane-3,4,5-triol;

(2S,3R,4S,5R)-2-((2S,3S,4R)-3,4-dihydroxy-2-(4-(7-phenylheptyl)-1H-1,2,3-triazol-1-yl)octadecyloxy)-6-(hydroxymethyl)- tetrahydro-28-pyrane-3,4,5-triol;

(2S,3R,4S,5R)-2-((2S,3S,4R)-3,4-dihydroxy-2-(4-(8-phenyloctyl)-1H-1,2,3-triazol-1-yl)octadecyloxy)-6-(hydroxymethyl)- tetrahydro-28-pyrane-3,4,5-triol;

11-amino-N-((2S,3S,4R)-3,4-dihydroxy-1-((2S,3R,4S,5R)-3,4,5-trihydroxy-6-(hydroxymethyl)-tetrahydro-28-pyran-2-yloxy)octadecan-2-yl)undecanamide;

12-amino-N-((2S,3S,4R)-3,4-dihydroxy-1-((2S,3R,4S,5R)-3,4,5-trihydroxy-6-(hydroxymethyl)-tetrahydro-2H-pyran-2-oxy)octadecan-2- yl)dodecanamide;

N-((2S,3S,4R)-3,4-dihydroxy-1-((2S,3R,4S,5R)-3,4,5-trihydroxy-6-(hydroxymethyl)-tetrahydro-2H-pyran-2-yloxy)octadecan-2-yl)-11- hydroxyundecanamide;

N-((2S,3S,4R)-3,4-dihydroxy-1-((2S,3R,4S,5R)-3,4,5-trihydroxy-6-(hydroxymethyl)-tetrahydro-2H-pyran-2-yloxy)octadecan-2-yl)-12- hydroxydodecanamide;

8-(diheptylamino)-N-((2S,3S,4R)-3,4-dihydroxy-1-((2S,3R,4S,5R)-3,4,5-trihydroxy-6-(hydroxymethyl)-tetrahydro-2H-pyran-2- yloxy)octadecan-2-yl)octanamide;

N-((2S,3S,4R)-3,4-dihydroxy-1-((2S,3R,4S,5R)-3,4,5-trihydroxy-6-(hydroxymethyl)-tetrahydro-2H-pyran-2-yloxy)octadecan-2-yl)-11- (dipentylamino)undecanamide;

11-(diheptylamino)-N-((2S,3S,4R)-3,4-dihydroxy-1-((2S,3R,4S,5R)-3,4,5-trihydroxy-6-(hydroxymethyl)-tetrahydro-2H-pyran-2- yloxy)octadecan-2-yl)undecanamide;

N-((2S,3S,4R)-3,4-dihydroxy-1-((2S,3R,4S,5R)-3,4,5-trihydroxy-6-(hydroxymethyl)-tetranydro-2H-pyran-2-yloxy)octadecan-2-yl)-11- mercaptoundecanamide;

N-((2S,3S,4R)-3,4-dihydroxy-1-((2S,3R,4S,5R)-3,4,5-dihydroxy-6-(hydroxymethyl)-tetrahydro-2Hpyran-2-yloxy)octadecan-2-yl)-12- mercaptododecanamide,

In some embodiments α-galactosylceramide compounds are pegylated. As used herein, the term “pegylated” refers to the conjugation of a compound moiety (i.e. α-galactosylceramide compound) with conjugate moiety(ies) containing at least one polyalkylene unit. In particular, the term pegylated refers to the conjugation of the compound moiety (i.e. α-galactosylceramide compound) with a conjugate moiety having at least one polyethylene glycol unit.

The present invention relates to a method for the prophylactic treatment of bacterial superinfections post-influenza in a subject in need thereof comprising the step of administrating said patient with therapeutically effective amount of at least one iNKT cell agonist.

By a “therapeutically effective amount” is meant a sufficient amount of an α-galactosylceramide compound to prevent bacterial superinfections post-influenza at a reasonable benefit/risk ratio applicable to any medical treatment.

It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiment, the iNKT cell agonist according to the invention is administered to the patient in combination with an anti-bacterial agent, such as antibiotics. Suitable antibiotics that could be coadministered in combination with the iNKT cell agonist according to the invention include, but are not limited to, at least one antibiotic selected from the group consisting of: ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid, amoxicillin, amoxicillin/clavulinic acid (Augmentin), cefuroxime,trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, and cephradine.

In some embodiment, the iNKT cell agonist according to the invention is administered to the patient in combination with an anti-inflammatory agent or an immunomodulator agent such as NSAIDs, aspirin, glucocorticoids, methotrexate, a Toll-Like receptor (i.e. TLR1, 2, 3, 4, 5, 6, 7, 8, or 9) agonists or antagonists, tumor necrosis factor alpha receptor (TNF-alpha) antagonists or inteleukin 1 (IL1) receptor antagonists. For example an TNF-alpha receptor antagonist may be a neutralizing (preferably non-depleting) anti-TNFα antibody such as adalimumab (Humira™) or Certolizumab pegol (Cimzia™)), and an IL-1 receptor antagonist may be anakinra (Kineret™)).

The α-galactosylceramide compounds may be combined with pharmaceutically acceptable excipients, and optionally sustained-release matrices, such as biodegradable polymers, to form pharmaceutical compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intranasal, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The α-galactosylceramide compound can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The pharmaceutical compositions may also be administered to the respiratory tract. The respiratory tract includes the upper airways, including the nose, oropharynx and larynx, followed by the lower airways, which include the trachea followed by bifurcations into the bronchi and bronchioli. Pulmonary delivery compositions can be delivered by inhalation by the patient of a dispersion so that the active ingredient within the dispersion can reach the lung where it can, for example, be readily absorbed through the alveolar region directly into blood circulation. Pulmonary delivery can be achieved by different approaches, including the use of nebulized, aerosolized, micellular and dry powder-based formulations; administration by inhalation may be oral and/or nasal. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self contained. Dry powder dispersion devices, for example, deliver drugs that may be readily formulated as dry powders. A pharmaceutical composition of the invention may be stably stored as lyophilized or spray-dried powders by itself or in combination with suitable powder carriers. The delivery of a pharmaceutical composition of the invention for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament. Examples of pharmaceutical devices for aerosol delivery include metered dose inhalers (MDIs), dry powder inhalers (DPIs), and air-jet nebulizers.

The α-galactosylceramide compound may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In some embodiments, the pharmaceutical composition comprises an anti-bacterial agent (e.g. an antibiotic) as above described.

The invention will be further illustrated by the following figures and examples. However, these examples and figures should not be interpreted in any way as limiting the scope of the present invention.

### FIGURES

FIG. 1. α-GalCer-mediated protection against S. pneumoniae challenge. Mice were infected (or not) with IAV (50 PFU) and 24 h before the S. pneumoniae challenge (1×10⁴ CFU) (day 7 post-influenza), they received α-GalCer (2 μg) or PBS as a control (intranasal route). The log-rank test for comparisons of Kaplan-Meier survival curves indicated a significant increase in the survival of α-GalCer-treated WT mice compared to PBS-treated mice. ***, p <0.001 (n=15 mice/group).

### EXAMPLE

Material & Methods

Reagents and Abs

α-GalCer was from Axxora Life Sciences (Coger, Paris, France). Monoclonal Abs against mouse CD5 (APC-conjugated), NK1.1 (PE- or PerCp-Cy5.5-conjugated), TCR-β (FITC- or V450-conjugated), CD69 (PerCp-Cy5.5-conjugated), CD45 (FITC or eFluor605NC-conjugated), CD11c (APC-conjugated), F4/80 (PE-Cy7-conjugated), MHC class II (Pacific-blue-conjugated), CD11b (Percp-Cy5.5-conjugated), Ly6C (AlexaFluor-700_conjugated), CCR2 (APC-conjugated), IFN-γ (AlexaFluor-488-conjugated), IL-17A (AlexaFluor-647-conjugated) and isotype controls were purchased from BD Pharmingen (Le Pont de Claix, France). The LIVE/DEAD® Fixable Dead Cell Stain Kit was purchased from Invitrogen (Cergy Pontoise, France). PE-conjugated PBS-57 glycolipid-loaded CD1d tetramer was from the NIAID Tetramer Facility (Emory University, Atlanta, Ga.).

Mice

Eight week-old male wild type (WT) C57BL/6 mice were purchased from Janvier (Le Genest-St-Isle, France). Jα18^{-/-} mice have been described in (25). For Influenza virus and S. pneumoniae infection, mice were maintained in a biosafety level 2 facility in the Animal Resource Center at the Pasteur Institute, Lille. All animal work conformed to the Pasteur Institute, Lille animal care and use committee guidelines (agreement number N° AF 16/20090 from the Comité d'Ethique en Expérimentation Animale Nord Pas-De-Calais).

Analysis of iNKT Activation

Lung mononuclear cells (MNC) were prepared as described (26). To analyze iNKT cells, MNC suspensions were incubated with appropriate dilutions of PE-conjugated PBS-57-loaded CD1d tetramer and V450-labelled anti-TCR-β for 30 min in PBS containing 2% FCS and 0.01% NaN₃. Cells were washed and fixed using IC Fixation Buffer (eBioscience, CliniSciences, Montrouge, France). Fixed cells were then permeabilized in Permeabilization Buffer (eBioscience), according to the manufacturer's instructions. Cells were washed and incubated with Alexa Fluor-488 and -647-conjugated mAb against IFN-γ or control rat IgG1 mAb in Permeabilization buffer. Cells were acquired and analyzed on a LSRFortessa™ (Becton Dickinson, Rungis, France) cytometer using the FACSDiva™ software.

Preparation and Adoptive Transfer of Pulmonary and Liver iNKT Cells

To purify iNKT cells, liver or lung MNCs were labelled with PE-conjugated PBS-57-loaded CD11d tetramer and FITC-conjugated anti-TCRβ Ab. After cell surface labelling, cells were sorted using a FACSAria (BD Biosciences). PBS57-loaded CD1d tetramer⁺ TCRβ⁺ cell purity after sorting was consistently >98%. Recipient mice were inoculated intravenously either with 1×10⁶ purified liver iNKT cells or intratrachealy with 3.5×10⁴ purified pulmonary iNKT cells. Control mice received the same volume of medium alone 16 h before IAV infection.

IAV Infection and Assessment of Gene Expression by Quantitative RT-PCR

Mice were anesthetized and administered intranasally with 50 μl of PBS containing 50 plaque forming unit (PFU) of virus (Scotland/20/74, H3N2) (27). Total RNAs from whole lungs of mock-treated or IAV-infected mice were extracted and cDNAs were synthesized by classical procedures. Quantitative RT-PCR was carried out in an ABI 7500 Thermocycler (Applied Biosystems, Foster City, Calif.) using 0.5 μM of specific primers and QuantiTect SYBR Green PCR Master Mix (Qiagen). PCR amplification of gapdh was performed to control for sample loading and to allow normalization between samples. ΔCt values were obtained by deducting the raw cycle threshold (Ct values) obtained for gapdh mRNA, the internal standard, from the Ct values obtained for investigated genes. For graphical representation, data are expressed as fold mRNA level increase compared to the expression level in mock-treated mice.

Infection with S. Pneumoniae and Assessment of Bacterial Counts

For S. pneumoniae serotype 1 (clinical isolate E1586) (28) infection, mice were anesthetized and administered i.n. with 50 μl of PBS containing the bacteria (1×10⁶ colony forming units, CFU). Mice were monitored daily for illness and mortality for a period of 7 days. Bacterial burden in the lungs was measured 24 h after infection by plating serial 10-fold dilutions of lung homogenates onto blood agar plates. The plates were incubated at 37° C. overnight and CFU were enumerated 24 h later.

Bacterial Surinfection Post-IAV

Mice, infected or not with IAV (50 PFU) 3, 7, 14 or 28 days earlier, were intranasally inoculated with 1×10⁴ S. pneumoniae serotype 1. The number of viable bacteria in the lungs was determined 24 h post S. pneumoniae challenge. For survival studies, mice were infected with 1×10⁴ S. pneumoniae 7 days after IAV infection. Mice were monitored daily for illness and mortality for a period of 15 days.

Analysis of Respiratory DCs and Inflammatory Monocytes

Mice were sacrified 7 days after IAV infection. Briefly, lung MNCs were initially labelled for dead cells with the Live/Dead cell viability kit according to the manufacturer's protocol. To allow DC identification, lung MNC were then labeled with appropriate dilutions of FITC-conjugated anti-CD45, APC-conjugated anti-CD11c, PE-Cy7-conjugated anti-F4/80 and Pacific-blue-conjugated anti MHC class II. For inflammatory monocyte/DC identification, MNC were labeled with FITC-conjugated anti-CD45, PE-Cy7-conjugated anti-F4/80, Percp-Cy5.5-conjugated CD11b, AlexaFluor-700-conjugated anti-Ly6C and APC-conjugated anti-CCR2. Then, cells were analysed on a LSR Fortessa (BD Biosciences) using FACSDiva software.

Neutralization of IL-10R

IAV-infected mice were injected i.p. with 1 mg of rat anti-IL-10R (1B1.3A) or with the isotype control mAb (HRPN) (BioXcell, West Lebanon, N.H.) 24 h prior to S. pneumoniae infection.

Statistical Analyses

Results are expressed as the mean ±SD or ±SEM. The statistical significance of differences between experimental groups was calculated by an ANOVA with a Bonferroni post test or an unpaired Student's t test (GraphPad Prism 5 Software, San Diego, USA). The appropriateness of using these parametric tests was assessed by checking if the population was Gaussian and the variance equal (Bartlett's test). Survival of mice was compared using Kaplan-Meier analysis and log-rank test. Results with a p value of less than 0.05 were considered significant.

Results

Respiratory Dendritic Cells Activate iNKT Cells to Control S. Pneumoniae Clearance Early After Infection

We first measured the activation status and the potential role of pulmonary iNKT cells during experimental S. pneumoniae serotype 1 infection. Relative to mock-treated mice, iNKT cells expressed more cell-surface CD69 and accumulated intracellular IFN-γ 36 h after S. pneumoniae challenge. In contrast, iNKT cells remained negative for IL-4 whilst only a small number of iNKT cells produced IL-17. Among APCs, DCs have been reported to be important in iNKT cell activation in many settings (12, 29). To investigate the potential role of respiratory DCs in the context of S. pneumoniae infection, transgenic CD11c.DTR mice were used. Treatment with diphtheria toxin strongly depleted respiratory DCs in the lung tissue and this resulted in a complete abrogation of iNKT cell activation in response to S. pneumoniae.

To address the natural role of iNKT cells in the control of S. pneumoniae, WT and Jα18^{-/-} mice were infected with S. pneumoniae and monitored daily for survival. Whilst at the dose used, 65% of WT animals survived to infection, almost all iNKT cell-deficient animals succumbed. This enhanced mortality was associated with a reduced capacity to clear S. pneumoniae in the lung tissue. Adoptive transfer of naïve iNKT cells into Jα18^{-/-} mice restored host defence mechanisms, as assessed by the determination of live bacteria in the lungs. Together, endogenous activation of iNKT cells by DCs is crucial to trigger early host defence mechanisms against S. pneumoniae.

iNKT Cells do not Play a Deleterious Role in Lethal synergism Between IAV and S. Pneumoniae.

Before studying the potential positive or negative role of iNKT cells in bacterial superinfection post influenza, we determined the optimal timing between exposure to the virus and exposure to the bacteria to achieve bacterial superinfection. For this, animals were infected with a mild, self-limiting dose of IAV (50 PFU) and were then superinfected with a sublethal dose of S. pneumoniae (1×10⁴ CFU) at different time points post-virus infection. Mice challenged with S. pneumoniae 7 days post-influenza had a high number of live bacteria in the lungs whilst animals infected after 3 days completely clear the bacteria. Of note, mice infected 2 and 4 weeks after IAV had a lower capacity to control bacterial replication but the numbers of bacteria in the lungs were lower relative to that found in mice receiving IAV 7 days earlier. For the rest of the study, S. pneumoniae was administered 7 days post IAV.

Among reasons explaining lethality during secondary bacterial infections that follow infection with IAV, the exacerbated pulmonary inflammation and tissue damages have been proposed (for review, (8)). Since iNKT cells can exert negative effect on lung inflammation in some pathological settings (20-24, 30, 31), we first examined the possibility that these cells could play a detrimental role in bacterial superinfection by enhancing pneumonia. At the doses used, single infections of WT mice and Jα18^{-/-} mice with IAV and S. pneumoniae did not result in mouse death. By contrast, when WT mice were infected with IAV and, 7 days later, with S. pneumoniae, the co-infected mice died between 2 and 5 days post-S. pneumoniae infection. Mortality correlated with the development of pneumonia and lung inflammation. Infection with the two agents did not significantly modify pneumonia and lung inflammation nor the rate and timing of mortality in Jα18^{-/-} mice, although a trend towards a more accelerated death was noticed in Jα18 ^{-/-} mice in some experiments. Analysis of CFU numbers indicated an increased, albeit not significant, enhancement of viable bacteria in Jα18^{-/-} mice. Thus, the absence of iNKT cells do not ameliorate the pathology associated with bacterial superinfection post-IAV challenge suggesting that these cells do not play a negative role in this setting.

Lung iNKT Cells from IAV-Experienced Mice Failed to Become Activated upon S. Pneumoniae Challenge

The experiment described above also suggested that iNKT cells do not play a positive role in the control of S. pneumoniae infection in IAV-experienced mice, a situation in contrast with naïve mice where iNKT cells clearly control the early development of S. pneumoniae. We first hypothesized that this could be due to a defected numbers of iNKT cells in the lungs at the time of S. pneumoniae infection. The number of pulmonary iNKT cells were not significantly modified 7 days after IAV infection. However, lung iNKT cells from IAV-experienced animals failed to be activated in response to S. pneumoniae challenge. Indeed, iNKT cells from double-infected animals did not produce IFN-γ whilst mice only infected with S. pneumoniae did so. Of interest, the lack of iNKT cell activation was associated with a dramatic reduced number of respiratory DCs in the lung tissue. On the other hand, a massive infiltration of inflammatory monocytes/DCs (CD11b^{hi} CCR2⁺ Ly6c⁺ CD11c^{+/-}) expressing a high level of CD1d was observed. Collectively, these data suggest that IAV infection impairs the capacity of iNKT cells to activate in response to S. pneumoniae challenge.

The transfer of Naïve iNKT Cells into IAV-Infected Mice Failed to Restore Host Defence Against S. Pneumoniae

Since pulmonary iNKT cells in mice previously infected with IAV are not functional in the context of S. pneumoniae infection, we investigated the possibility that transfer of fresh iNKT cells could restore host defence mechanisms against S. pneumoniae. Adoptive transfer of naïve lung iNKT cells to the airway of prior IAV-infected mice did not restore influenza-impaired antibacterial host defence as the numbers of bacteria were not decreased in transferred mice. Moreover, mice transferred with hepatic iNKT cells by the i.v. route failed to promote efficient immunity against S. pneumoniae. These experiments suggested that local immuno-suppressive mechanisms able to curtail iNKT cell activation upon S. pneumoniae challenge develop in the lung of IAV-infected mice.

Expression of IL-10 Favours Bacterial Superinfection Possibly by Abrogating IFN-γ Release by iNKT Cells

Among potential immunosuppressive candidates, we first focused on IL-10, a cytokine described to play a negative role in bacterial superinfection post H1N1 influenza (32, 33). The expression of IL-10 mRNA peaked 7 days after IAV, a time point that coincides with the peak of bacterial susceptibility. More importantly, the use of anti-IL-10R Ab partially restored the ability of H3N2 IAV-infected mice to defend against S. pneumoniae. Indeed, the survival rate of anti-IL-10R-treated double infected mice was enhanced by ˜60% relative to mice treated with the isotype control. Likewise, blockade of IL-10R functions resulted in a reduced number of bacteria in the lung tissue.

To establish a potential link between IL-10 production during influenza and the lack of iNKT cell activation after S. pneumoniae challenge, IAV-infected mice were treated with anti-IL-10R Ab just before S. pneumoniae infection. Remarkably, this treatment resulted in the complete restoration of iNKT cell activation in terms of IFN-γ production. Collectively, these data demonstrate that IL-10 is the main factor preventing the activation of pulmonary iNKT cells in IAV experiencing animals, a phenomenon that might lead to enhanced susceptibility to bacterial infection post-influenza.

Exogenous Activation of iNKT Cells by α-GalCer Protects Against Bacterial Superinfection Post Influenza

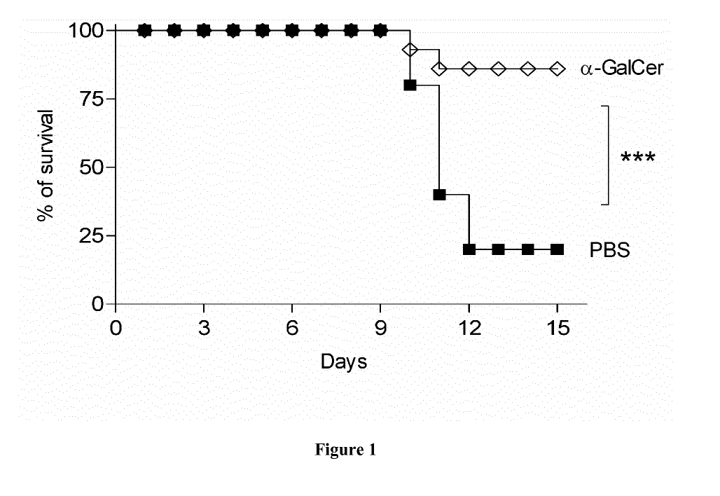

We and others have shown that exogenous activation of lung iNKT cells with α-GalCer leads to the clearance of S. pneumoniae after infection with a high dose of bacteria (12, 34). The potential protective effect of α-GalCer in the context of previous influenza infection has not yet been studied. We first investigated whether the lung immunosuppressive environment dampens iNKT cell activation in response to α-GalCer. iNKT cells from IAV-experienced animals produced IFN-γ to a similar level than iNKT cells from naïve animals. Moreover, administration of α-GalCer protected by ˜70% against S. pneumoniae challenge, as assessed by the enhanced survival rate (FIG. 1). Together, exogenous activation of iNKT by α-GalCer reverses the immunosuppresive environment in the lungs imposed by IAV and protects against bacterial superinfection.

Discussion:

Secondary bacterial infection often occurs after pulmonary IAV infection and is a common cause of severe disease in humans, yet the mechanisms responsible for this viral-bacterial synergy in the lung are only poorly understood. It is therefore critical that we reach a better understanding of the causes and potential treatments for post-IAV bacterial superinfection. Alteration of respiratory mucosal innate immunity in IAV-experienced hosts is a cardinal feature leading to heightened susceptibility to respiratory bacteria (8). Although macrophages, neutrophils and NK cell dysfunctions have been described to account for enhanced susceptibility to bacterial superinfection (35-38), the precise mechanisms leading to the altered innate response to S. pneumoniae are not entirely resolved. Post-viral desensitization of some innate sensors, such as Toll-like receptors (39), as well as overt expression of certain cytokines including IFN type I, IFN-γ and IL-10 (32, 33, 36, 37, 40) play a major role in this setting. Here, we show that IAV infection leads to increased susceptibility to subsequent bacterial superinfection by impairing iNKT cell response in the lung, a phenomenon that depended on IL-10. We also provide evidence that α-GalCer overcomes the immunosuppressive state in the lungs, thus protecting against bacterial superinfection.

The positive role of iNKT cells in the control of S. pneumoniae (serotype 3) infection has already been described (34, 41-43). Here, we first re-evaluated the natural role of iNKT cells using another major serotype in humans, S. pneumoniae serotype 1. First, in agreement with (42, 43), we observed that lung iNKT cells become activated (IFN-γ and to a lower extent −17) early after S. pneumoniae infection. Our recent findings have highlighted the role of respiratory DCs in the activation of pulmonary iNKT cells in response to the canonical activator α-GalCer (51). We herein show that respiratory DCs are also critical players in iNKT cell activation in the context of S. pneumoniae challenge. Interestingly, iNKT cells are important to rapidly clear bacteria in the lung tissue upon sublethal infection, a phenomenon that accounts for the survival. Attempts are in progress to study the mechanisms of iNKT cell-mediated protection against sublethal S. pneumoniae infection. It is likely that iNKT cell-derived IFN-γ, shown to be critical in macrophage activation and in neutrophil-mediated clearance of respiratory bacteria (41, 44), plays a critical part in this setting.

We next studied whether iNKT cells could play a role during bacterial superinfection. To this end, we have established a murine model of heterologous infections by an H3N2 IAV and S. pneumoniae. Severe sickness and heightened bacterial infection in flu and S. pneumoniae dual-infected animals were associated with severe immunopathology in the lung followed by death. Since IFN-γ was described as a detrimental factor favouring bacterial superinfection (36), and because iNKT cells strongly contribute to IFN-γ production in the lungs during IAV infection (26), we first hypothesized that iNKT cells could be detrimental during bacterial superinfection. This hypothesis was also based on the fact that iNKT cells sometimes participate in lung inflammation (21, 22, 24, 30, 31). Our data showed that Jα18^{-/-} mice still succumbed to IAV/S. pneumoniae co-infection with kinetics that were similar to those observed in WT animals. Thus, iNKT cells are not deleterious in this system. Whilst in double-infected animals, iNKT cells do not favour the pathology, the lack of iNKTs did not result in increased susceptibility to S. pneumoniae. Analysis of iNKT cell number revealed no disappearance of these cells in IAV-infected mice, a phenomenon that could have occurred in response to enhanced apoptosis (45-47). Strikingly, resident iNKT cells from virus infected mice became refractory to secondary stimulation as they failed to produce IFN-γ in response to S. pneumoniae. Furthermore, adoptive transfer of naïve iNKT cells was not sufficient to restore iNKT cell functions in the lungs and did not prevent bacterial superinfection. This suggests that mechanisms underlining iNKT cell activation during S. pneumoniae challenge are not operative in the lung environment from IAV-experienced animals. Among potential immunosuppressive molecules, IL-10 has been recently described as an important factor involved in bacterial superinfection (32), although this finding has recently been called into question (36). Analysis of IL-10 transcript expression indicated a strong enhancement 7 days post infection, a time point that coincides with the peak of bacterial infection susceptibility. In agreement with (32), we confirmed that neutralizing IL-10 activities (herein by mean of anti-IL10R Ab) partially rescued the inappropriate immune response in the lungs, thus leading to an enhanced survival rate and to a higher rate of S. pneumoniae clearance in the lung tissue. Of importance, IL-10R neutralisation during IAV infection also restored iNKT cell activation in the lungs after S. pneumoniae challenge. It is likely that the defected iNKT cell function in flu-experienced animals is probably an upstream mechanism of depressed antibacterial activities by alveolar macrophages or neutrophils, known to be important in anti-pneumococcal innate immunity (48-50). Natural Killer cells, known to be trans-activated by iNKT cells, may also be important in this setting.

We show that the abrogated activation of iNKT cells in flu-infected mice correlates with IL-10 synthesis and with heightened bacterial load and mortality during secondary respiratory infection. Our data therefore suggests that defected iNKT cell response may be one possible contributor to the common secondary bacterial pneumonia associated with pandemic and seasonal influenza infection. We thus investigated whether exogenous activation of iNKT cells by means of α-GalCer treatment, just before S. pneumoniae challenge, can relieve inhibitory mechanisms that developed in the lungs of IAV-infected animals. We show that i.n. administration of α-GalCer leads to iNKT cell activation. This shows that pulmonary iNKT cells are not intrinsically “anergic” 1 week after sublethal IAV infection, unlike after a lethal challenge (26). Since at day 7 p.i., respiratory DCs are not detectable in the lung tissue, it is likely that neorecruited APC (particularly inflammatory monocytes/DCs), rather that resident APC, play a role in iNKT cell activation in response to α-GalCer. Importantly, we show that α-GalCer treatment protects against bacterial superinfection as assessed by the dramatically enhanced survival rate of co-infected mice. This indicates that the alteration of the effector functions of innate immune cells (i.e.

macrophages and neutrophils) is not irreversible and that factors released by iNKT are sufficient to overcome local immunosuppression. Even if antibiotic may be a viable option for treatment against bacterial superinfections, with the ever-increasing problem of antibiotic resistance, immunomodulation might be an attractive alternative to control bacterial superinfection. In this setting, the use of α-GalCer analogues offers a promising approach that could be exploited in the human systems, particularly during epidemics.

### REFERENCES

Throughout this application, various references describe the state of the art to which this invention pertains. The disclosures of these references are hereby incorporated by reference into the present disclosure.
1. Palese, P. 2004. Influenza: old and new threats. Nat Med 10:S82-87.
2. White, M. R., M. Doss, P. Boland, T. Tecle, and K. L. Hartshorn. 2008. Innate immunity to influenza virus: implications for future therapy. Expert Rev Clin Immunol 4:497-514.
3. McGill, J., J. W. Heusel, and K. L. Legge. 2009. Innate immune control and regulation of influenza virus infections. J Leukoc Biol 86:803-812.
4. Brundage, J. F. 2006. Interactions between influenza and bacterial respiratory pathogens: implications for pandemic preparedness. Lancet Infect Dis 6:303-312.
5. Beadling, C., and M. K. Slifka. 2004. How do viral infections predispose patients to bacterial infections? Curr Opin Infect Dis 17:185-191.
6. Hartshorn, K. L. New look at an old problem: bacterial superinfection after influenza. Am J Pathol 176:536-539.
7. Brundage, J. F., and G. D. Shanks. 2008. Deaths from bacterial pneumonia during 1918-19 influenza pandemic. Emerg Infect Dis 14:1193-1199.
8. Snelgrove, R. J., A. Godlee, and T. Hussell. Airway immune homeostasis and implications for influenza-induced inflammation. Trends Immunol 32:328-334.
9. Goulding, J., R. Snelgrove, J. Saldana, A. Didierlaurent, M. Cavanagh, E. Gwyer, J. Wales, E. L. Wissinger, and T. Hussell. 2007. Respiratory infections: do we ever recover? Proc Am Thorac Soc 4:618-625.
10. McCullers, J. A. 2006. Insights into the interaction between influenza virus and pneumococcus. Clin Microbiol Rev 19:571-582.
11. Lee, L. N., P. Dias, D. Han, S. Yoon, A. Shea, V. Zakharov, D. Parham, and S. R. Sarawar. A mouse model of lethal synergism between influenza virus and Haemophilus influenzae. Am J Pathol 176:800-811.
12. Tupin, E., Y. Kinjo, and M. Kronenberg. 2007. The unique role of natural killer T cells in the response to microorganisms. Nat Rev Microbiol 5:405-417.
13. Cohen, N. R., S. Garg, and M. B. Brenner. 2009. Antigen Presentation by CD1 Lipids, T Cells, and NKT Cells in Microbial Immunity. Adv Immunol 102:1-94.
14. Tessmer, M. S., A. Fatima, C. Paget, F. Trottein, and L. Brossay. 2009. NKT cell immune responses to viral infection. Expert Opin Ther Targets 13:153-162.
15. Barral, D. C., and M. B. Brenner. 2007. CD1 antigen presentation: how it works. Nat Rev Immunol 7:929-941.
16. Bendelac, A., P. B. Savage, and L. Teyton. 2007. The biology of NKT cells. Annu Rev Immunol 25:297-336.
17. Cerundolo, V., J. D. Silk, S. H. Masri, and M. Salio. 2009. Harnessing invariant NKT cells in vaccination strategies. Nat Rev Immunol 9:28-38.
18. Godfrey, D. I., and M. Kronenberg. 2004. Going both ways: immune regulation via CD1d-dependent NKT cells. J Clin Invest 114:1379-1388.
19. Van Kaer, L., and S. Joyce. 2005. Innate immunity: NKT cells in the spotlight. Curr Biol 15:R429-431.
20. Kim, E. Y., J. T. Battaile, A. C. Patel, Y. You, E. Agapov, M. H. Grayson, L. A. Benoit, D. E. Byers, Y. Alevy, J. Tucker, S. Swanson, R. Tidwell, J. W. Tyner, J. D. Morton, M. Castro, D. Polineni, G. A. Patterson, R. A. Schwendener, J. D. Allard, G. Peltz, and M. J. Holtzman. 2008. Persistent activation of an innate immune response translates respiratory viral infection into chronic lung disease. Nat Med 14:633-640.
21. Lisbonne, M., S. Diem, A. de Castro Keller, J. Lefort, L. M. Araujo, P. Hachem, J. M. Fourneau, S. Sidobre, M. Kronenberg, M. Taniguchi, P. Van Endert, M. Dy, P. Askenase, M. Russo, B. B. Vargaftig, A. Herbelin, and M. C. Leite-de-Moraes. 2003. Cutting edge: invariant V alpha 14 NKT cells are required for allergen-induced airway inflammation and hyperreactivity in an experimental asthma model. J Immunol 171:1637-1641.
22. Akbari, O, P. Stock, E. Meyer, M. Kronenberg, S. Sidobre, T. Nakayama, M. Taniguchi, M. J. Grusby, R. H. DeKruyff, and D. T. Umetsu. 2003. Essential role of NKT cells producing IL-4 and IL-13 in the development of allergen-induced airway hyperreactivity. Nat Med 9:582-588.
23. Pichavant, M., S. Goya, E. H. Meyer, R. A. Johnston, H. Y. Kim, P. Matangkasombut, M. Zhu, Y. Iwakura, P. B. Savage, R. H. DeKruyff, S. A. Shore, and D. T. Umetsu. 2008. Ozone exposure in a mouse model induces airway hyperreactivity that requires the presence of natural killer T cells and IL-17. J Exp Med 205:385-393.
24. Sharma, A. K., D. J. LaPar, Y. Zhao, L. Li, C. L. Lau, I. L. Kron, Y. Iwakura, M. D. Okusa, and V. E. Laubach. Natural killer T cell-derived IL-17 mediates lung ischemia-reperfusion injury. Am J Respir Crit Care Med 183:1539-1549.
25. Cui, J., T. Shin, T. Kawano, H. Sato, E. Kondo, I. Toura, Y. Kaneko, H. Koseki, M. Kanno, and M. Taniguchi. 1997. Requirement for Valpha14 NKT cells in IL-12-mediated rejection of tumors. Science 278:1623-1626.
26. Paget, C., S. Ivanov, J. Fontaine, F. Blanc, M. Pichavant, J. Renneson, E. Bialecki, J. Pothlichet, C. Vendeville, G. Barba-Spaeth, M. R. Huerre, C. Faveeuw, M. Si-Tahar, and F. Trottein. Potential role of invariant NKT cells in the control of pulmonary inflammation and CD8+ T cell response during acute influenza A virus H3N2 pneumonia. J Immunol 186:5590-5602.
27. Guillot, L., R. Le Goffic, S. Bloch, N. Escriou, S. Akira, M. Chignard, and M. Si-Tahar. 2005. Involvement of toll-like receptor 3 in the immune response of lung epithelial cells to double-stranded RNA and influenza A virus. J Biol Chem 280:5571-5580.
28. Tomasz, A., A. Corso, E. P. Severina, G. Echaniz-Aviles, M. C. Brandileone, T. Camou, E. Castaneda, O. Figueroa, A. Rossi, and J. L. Di Fabio. 1998. Molecular epidemiologic characterization of penicillin-resistant Streptococcus pneumoniae invasive pediatric isolates recovered in six Latin-American countries: an overview. PAHO/Rockefeller University Workshop. Pan American Health Organization. Microbial drug resistance (Larchmont, N.Y. 4:195-207.
29. Bai, L., M. G. Constantinides, S. Y. Thomas, R. Reboulet, F. Meng, F. Koentgen, L. Teyton, P. B. Savage, and A. Bendelac. Distinct APCs explain the cytokine bias of alpha-galactosylceramide variants in vivo. J Immunol 188:3053-3061.
30. Wingender, G., P. Rogers, G. Batzer, M. S. Lee, D. Bai, B. Pei, A. Khurana, M. Kronenberg, and A. A. Horner. Invariant NKT cells are required for airway inflammation induced by environmental antigens. J Exp Med 208:1151-1162.
31. Kim, J. H., and D. H. Chung. CD1d-restricted IFN-gamma-secreting NKT cells promote immune complex-induced acute lung injury by regulating macrophage-inflammatory protein-1 alpha production and activation of macrophages and dendritic cells. J Immunol 186:1432-1441.
32. van der Sluijs, K. F., L. J. van Elden, M. Nijhuis, R. Schuurman, J. M. Pater, S. Florquin, M. Goldman, H. M. Jansen, R. Lutter, and T. van der Poll. 2004. IL-10 is an important mediator of the enhanced susceptibility to pneumococcal pneumonia after influenza infection. J Immunol 172:7603-7609.
33. van der Sluijs, K. F., M. Nijhuis, J. H. Levels, S. Florquin, A. L. Mellor, H. M. Jansen, T. van der Poll, and R. Lutter. 2006. Influenza-induced expression of indoleamine 2,3-dioxygenase enhances interleukin-10 production and bacterial outgrowth during secondary pneumococcal pneumonia. J Infect Dis 193:214-222.
34. Kawakami, K., N. Yamamoto, Y. Kinjo, K. Miyagi, C. Nakasone, K. Uezu, T. Kinjo, T. Nakayama, M. Taniguchi, and A. Saito. 2003. Critical role of Valpha14+ natural killer T cells in the innate phase of host protection against Streptococcus pneumoniae infection. Eur J Immunol 33:3322-3330.
35. McNamee, L. A., and A. G. Harmsen. 2006. Both influenza-induced neutrophil dysfunction and neutrophil-independent mechanisms contribute to increased susceptibility to a secondary Streptococcus pneumoniae infection. Infect Immun 74:6707-6721.
36. Sun, K., and D. W. Metzger. 2008. Inhibition of pulmonary antibacterial defense by interferon-gamma during recovery from influenza infection. Nat Med 14:558-564.
37. Shahangian, A., E. K. Chow, X. Tian, J. R. Kang, A. Ghaffari, S. Y. Liu, J. A. Belperio, G. Cheng, and J. C. Deng. 2009. Type I IFNs mediate development of postinfluenza bacterial pneumonia in mice. J Clin Invest 119:1910-1920.
38. Small, C. L., C. R. Shaler, S. McCormick, M. Jeyanathan, D. Damjanovic, E. G. Brown, P. Arck, M. Jordana, C. Kaushic, A. A. Ashkar, and Z. Xing. Influenza infection leads to increased susceptibility to subsequent bacterial superinfection by impairing NK cell responses in the lung. J Immunol 184:2048-2056.
39. Didierlaurent, A., J. Goulding, S. Patel, R. Snelgrove, L. Low, M. Bebien, T. Lawrence, L. S. van Rijt, B. N. Lambrecht, J. C. Sirard, and T. Hussell. 2008. Sustained desensitization to bacterial Toll-like receptor ligands after resolution of respiratory influenza infection. J Exp Med 205:323-329.
40. Kudva, A., E. V. Scheller, K. M. Robinson, C. R. Crowe, S. M. Choi, S. R. Slight, S. A. Khader, P. J. Dubin, R. I. Enelow, J. K. Kolls, and J. F. Alcorn. Influenza A inhibits Th17-mediated host defense against bacterial pneumonia in mice. J Immunol 186:1666-1674.
41. Nakamatsu, M., N. Yamamoto, M. Hatta, C. Nakasone, T. Kinjo, K. Miyagi, K. Uezu, K. Nakamura, T. Nakayama, M. Taniguchi, Y. Iwakura, M. Kaku, J. Fujita, and K. Kawakami. 2007. Role of interferon-gamma in Valpha14+ natural killer T cell-mediated host defense against Streptococcus pneumoniae infection in murine lungs. Microbes Infect 9:364-374.
42. Kinjo, Y., P. Illarionov, J. L. Vela, B. Pei, E. Girardi, X. Li, Y. Li, M. Imamura, Y. Kaneko, A. Okawara, Y. Miyazaki, A. Gomez-Velasco, P. Rogers, S. Dahesh, S. Uchiyama, A. Khurana, K. Kawahara, H. Yesilkaya, P. W. Andrew, C. H. Wong, K. Kawakami, V. Nizet, G. S. Besra, M. Tsuji, D. M. Zajonc, and M. Kronenberg. Invariant natural killer T cells recognize glycolipids from pathogenic Gram-positive bacteria. Nat Immunol 12:966-974.
43. Brigl, M., R. V. Tatituri, G. F. Watts, V. Bhowruth, E. A. Leadbetter, N. Barton, N. R. Cohen, F. F. Hsu, G. S. Besra, and M. B. Brenner. Innate and cytokine-driven signals, rather than microbial antigens, dominate in natural killer T cell activation during microbial infection. J Exp Med 208:1163-1177.
44. Nieuwenhuis, E. E., T. Matsumoto, M. Exley, R. A. Schleipman, J. Glickman, D. T.

Bailey, N. Corazza, S. P. Colgan, A. B. Onderdonk, and R. S. Blumberg. 2002. CD1d-dependent macrophage-mediated clearance of Pseudomonas aeruginosa from lung. Nat Med 8:588-593.
45. Leite-de-Moraes, M. C., A. Herbelin, C. Gouarin, Y. Koezuka, E. Schneider, and M. Dy. 2000. Fas/Fas ligand interactions promote activation-induced cell death of NK T lymphocytes. J Immunol 165:4367-4371.
46. Herold, S., M. Steinmueller, W. von Wulffen, L. Cakarova, R. Pinto, S. Pleschka, M. Mack, W. A. Kuziel, N. Corazza, T. Brunner, W. Seeger, and J. Lohmeyer. 2008. Lung epithelial apoptosis in influenza virus pneumonia: the role of macrophage-expressed TNF-related apoptosis-inducing ligand. J Exp Med 205:3065-3077.
47. McLean, J. E., E. Datan, D. Matassov, and Z. F. Zakeri. 2009. Lack of Bax prevents influenza A virus-induced apoptosis and causes diminished viral replication. J Virol 83:8233-8246.
48. Buttery, J., and E. R. Moxon. 2002. Capsulate bacteria and the lung. Br Med Bull 61:63-80.
49. Garvy, B. A., and A. G. Harmsen. 1996. The importance of neutrophils in resistance to pneumococcal pneumonia in adult and neonatal mice. Inflammation 20:499-512.
50. Kadioglu, A., and P. W. Andrew. 2004. The innate immune response to pneumococcal lung infection: the untold story. Trends Immunol 25:143-149.
51. Ivanov S, Fontaine J, Paget C, Macho Fernandez E, Van Maele L, Renneson J, Maillet I, Wolf N M, Rial A, Léger H, Ryffel B, Frisch B, Chabalgoity J A, Sirard J C, Benecke A, Faveeuw C, Trottein F. Key Role for Respiratory CD103+ Dendritic Cells, IFN-γ, and IL-17 in Protection Against Streptococcus pneumoniae Infection in Response to α-Galactosylceramide. J Infect Dis. 2012 Sep;206(5):723-34. Epub 2012 Jun 21.

1. A method for the prophylactic treatment of bacterial superinfections post-influenza in a subject in need thereof comprising administering to the subject a therapeutically effective amount of an invariant Natural Killer T (iNKT) cell agonist.

2. The method of claim 1 wherein the iNKT cell agonist is an α-galactosylceramide compound.

3. The method of claim 1 wherein the influenza infection is associated with Influenza virus A or B.

4. The method of claim 3 wherein the influenza infection is caused by an influenza virus A that is H1N1, H2N2, H3N2 or H5N1.

5. The method according to claim 1 wherein the bacterial superinfection is selected from the group consisting of lower respiratory tract infections, middle ear infections and bacterial sinusitis.

6. The method according to claim 1 wherein the bacterial superinfection may be mediated by at least one organism selected from the group consisting of Streptococcus pneumoniae; Staphylococcus aureus; Haemophilus influenza, Mycoplasma species and Moraxella catarrhalis.

7. The method according to claim 1 wherein the subject is selected from the group consisting of subjects who are at least 50 years old, subjects who reside in chronic care facilities, subjects who have chronic disorders of the pulmonary or cardiovascular system, subjects who required regular medical follow-up or hospitalization during the preceding year because of chronic metabolic diseases, renal dysfunction, hemoglobinopathies, or immunosuppression, children less than 14 years of age, patients between 6 months and 18 years of age who are receiving long-term aspirin therapy, and women who will be in the second or third trimester of pregnancy during the influenza season.

8. The method according to claim 1 which is suitable for the prevention of bacterial superinfection post-influenza in subjects older than 1 year old and less than 14 years old, subjects between the ages of 50 and 65, and adults who are older than 65 years of age.

9. The method according to claim 1 wherein the iNKT cell agonist is administered to the patient in combination with an anti-bacterial agent.

10. The method of claim 12 wherein the antibiotic is selected from the group consisting of ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid, amoxicillin, amoxicillin/clavulinic acid (Augmentin), cefuroxime,trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, and cephradine.

11. The method according to claim 1 wherein the iNKT cell agonist is administered to the respiratory tract.

12. The method of claim 9, wherein said anti-bacterial agent is an antibiotic.
